(19) United States
(12) Patent Application Publication
(10) Pub. No.: US 2007/0118026 A1
(43) Pub. Date: May 24, 2007

(54) APPARATUS, SYSTEM, AND METHOD FOR LIGHTING CONTROL, AND COMPUTER PROGRAM PRODUCT

(75) Inventors: Kenichi Kameyama, Minato-ku, Tokyo (JP); Takuji Suzuki, Minato-ku, Tokyo (JP); Kazushige Ouchi, Minato-ku, Tokyo (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: KABUSHIKI KAISHA TOSHIBA, Minato-ku (JP)

(21) Appl. No.: 11/556,721

(22) Filed: Nov. 6, 2006

(30) Foreign Application Priority Data
Nov. 9, 2005 (JP) 2005-325389

(51) Int. Cl. A61B 5/00

(52) U.S. Cl. 600/300

(57) ABSTRACT

A detecting unit detects a sleep state of a user. If the user is awakened halfway through his/her sleep, a control unit controls a lighting device to irradiate a light in a waveband with relatively small melatonin-production suppression effect.

### CROSS-REFERENCE TO RELATED APPLICATIONS

This application is based upon and claims the benefit of priority from the prior Japanese Patent Application No. 2005-325389, filed on Nov. 9, 2005; the entire contents of which are incorporated herein by reference.

### BACKGROUND OF THE INVENTION

1. Field of the Invention

The present invention generally relates to lighting control, and particularly relates to control lighting of a lighting device.

2. Description of the Related Art

Various methods are known for determining a user's sleep state and a user's environmental state, and then controlling bedroom lighting based on the determined states. For example, there is known a method of controlling lighting for a user by (1) turning off a light when it is detected that the user falls asleep, (2) gradually increasing illuminance and color temperature as time is closer to user's wakeup time so that the user wakes up efficiently and in good mood. A related art has been disclosed in “Sleep Consulting and Experience—Room Lighting System”, Matsushita Electric Works, Ltd. Technical Report Vol. 53, No. 1, pp. 33-38.

It is already known that a change in the luminance conditions around a person affects sleep of the person. However, no considerations have been given as to how the wavelength of the light affects the sleep. At present, therefore, only the physiological effect or psychological effect of the luminance is available for lighting control.

Because of no considerations to wavelength characteristics of a light source, a user is often awaken halfway through his/her sleep or exposed to light incompatible with physiological reaction before sleep onset, after sleep onset, at wakeup time or the like. This often, disadvantageously causes the user to suffer insomnia.

Moreover, it is common that one bedroom is used by a plurality of users such as a husband and a wife or the members of a family. In this case, if one of the users turns on light, the light disadvantageously affects not only the user who turns on the light but also the other user or users who have already fallen asleep.

### SUMMARY OF THE INVENTION

According to an aspect of the present invention, a lighting control apparatus includes a detecting unit that detects a sleep state of a user; and a control unit that controls a lighting device to irradiate a light in a waveband with a relatively small melatonin-production suppressing effect onto the user if the detecting unit detects a state before a sleep onset.

According to another aspect of the present invention, a lighting system includes the above lighting control apparatus according to claim 1; and the lighting device that irradiates the light in a waveband with a relatively small melatonin-production suppressing effect onto the user.

According to still another aspect of the present invention, a lighting control apparatus includes a detecting unit that detects a sleep state of a user; and a control unit that controls a lighting device to irradiate a light in a waveband corresponding to scotopia if the detecting unit detects that the user is awakened halfway through user's sleep.

According to still another aspect of the present invention, a lighting control apparatus includes a detecting unit that detects sleep states of a plurality of users; and a control unit that controls a lighting device to irradiate a light of which the users are less sensible through eyelids of the users if the sleep-state detecting unit detects that at least one of the users is awake and that remaining users are asleep.

According to still another aspect of the present invention, a method for lighting control includes detecting a sleep state of a user; and controlling a lighting device to irradiate a light in a waveband with a relatively small melatonin-production suppressing effect if a state before a sleep onset is detected at the detecting.

According to still another aspect of the present invention, a computer program product comprising a computer-readable recording medium, the computer-readable recording medium executable by a computer and including a plurality of instructions for a lighting control processing, the instructions including an instruction to detect a sleep state of a user; and an instruction to control a lighting device to irradiate a light in a waveband with a relatively small melatonin-production suppressing effect if a state before a sleep onset is detected.

### BRIEF DESCRIPTION OF THE DRAWINGS

FIG. 1 is a schematic of an overall light control system according to an embodiment of the present invention;

FIG. 2 is a schematic for explaining how a sleep sensor shown in FIG. 1 can be attached to a user;

FIG. 3 is a detailed functional block diagram of the sleep sensor;

FIG. 4 is a detailed functional block diagram of a lighting control apparatus shown in FIG. 1;

FIG. 5 is a graph for explaining a processing performed by an autonomic-nerve-index calculating unit shown in FIG. 4;

FIG. 6 is a table for explaining lighting control exercised when only one person is going to sleep in a bedroom;

FIG. 7 is a table for explaining lighting control exercised when a plurality of persons is going to sleep in one bedroom;

FIG. 8 is a graph of the relationship between wavelength of light and human sensitivity to light;

FIG. 9 is a flowchart of a lighting control processing performed by the lighting control system shown in FIG. 1;

FIG. 10 is a flowchart of a sleep-state determination processing shown in FIG. 9; and

FIG. 11 is a hardware block diagram of the lighting control apparatus shown in FIG. 1.

### DETAILED DESCRIPTION OF THE INVENTION

Exemplary embodiments of the present invention will be explained hereinafter in detail with reference to the accompanying drawings. It is to be noted that the present invention is not limited by the embodiments.

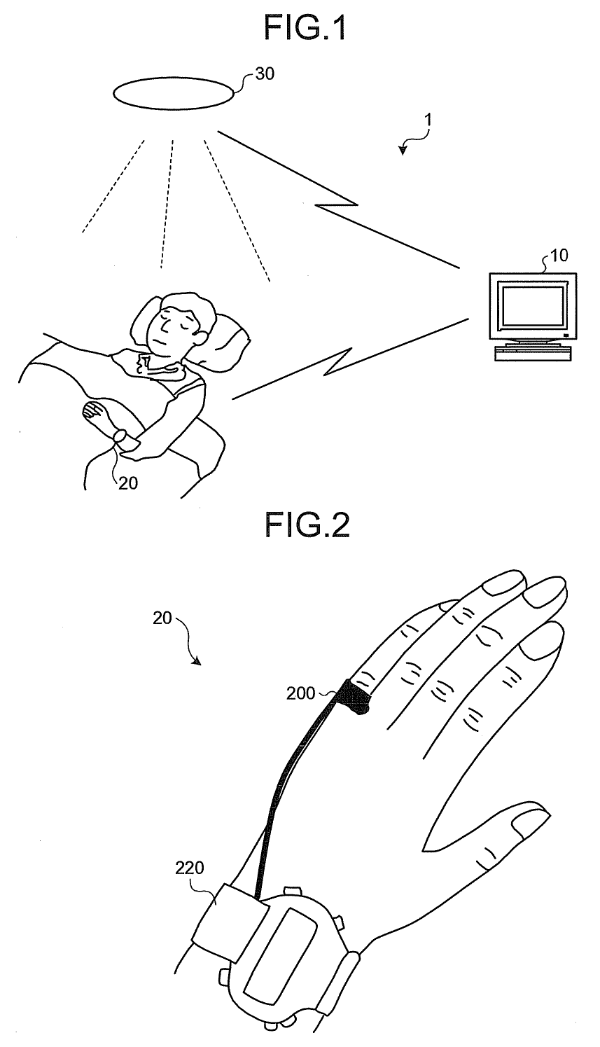

As shown in FIG. 1, a lighting control system 1 according to an embodiment of the present invention includes a lighting control apparatus 10, a sleep sensor 20, and a lighting device 30.

The sleep sensor 20 measures the pulse wave of a user to which the sleep sensor 20 is attached, and transmits the measurement result to the lighting control apparatus 10. The sleep sensor 20 can be configured to transmit data to the lighting control apparatus 10 through wireless communication using, for example Bluetooth. Alternatively, the sleep sensor 20 can be configured to transmit data to the lighting control apparatus 10 through wired communication. The lighting control apparatus 10 determines the sleep state of the user based on the measurement result relating to the pulse wave. The sleep state includes a state before sleep onset, an asleep state in which the user is asleep, a state at and after the sleep onset, and an awakened state halfway through his/her sleep. The asleep states include a rapid eye movement (REM) sleep (state) and a non-REM sleep (state).

The lighting control apparatus 10 controls the lighting device 30 according to the user's sleep state. The lighting device 30 irradiates light under control of the lighting control apparatus 10.

The lighting device 30 is a light source. The intensity and the waveband of the light emitted by the lighting device 30 are variable and can be controlled as desired. The lighting device 30 can be constituted by a plurality of light-emitting diodes (LEDs) or a laser diode (LD). Alternatively, the lighting device 30 can be constituted by a combination of an optical filter, transmittance-wavelength characteristics of which can be changed, and an incandescent lamp. A fluorescent lamp or LEDs can be used instead of the incandescent lamp. The optical filter, the transmittance-wavelength characteristics of which can be changed, can be realized by, for example, switching over a plurality of optical filters with hardware.

The sleep sensor 20 includes a sensor 200 and an operation unit 220. As shown in FIG. 2, the operation unit 220 is attached to, like a wristwatch, the user's wrist. The sensor 200 is attached to the user's little finger and it measures the pulse wave of the user.

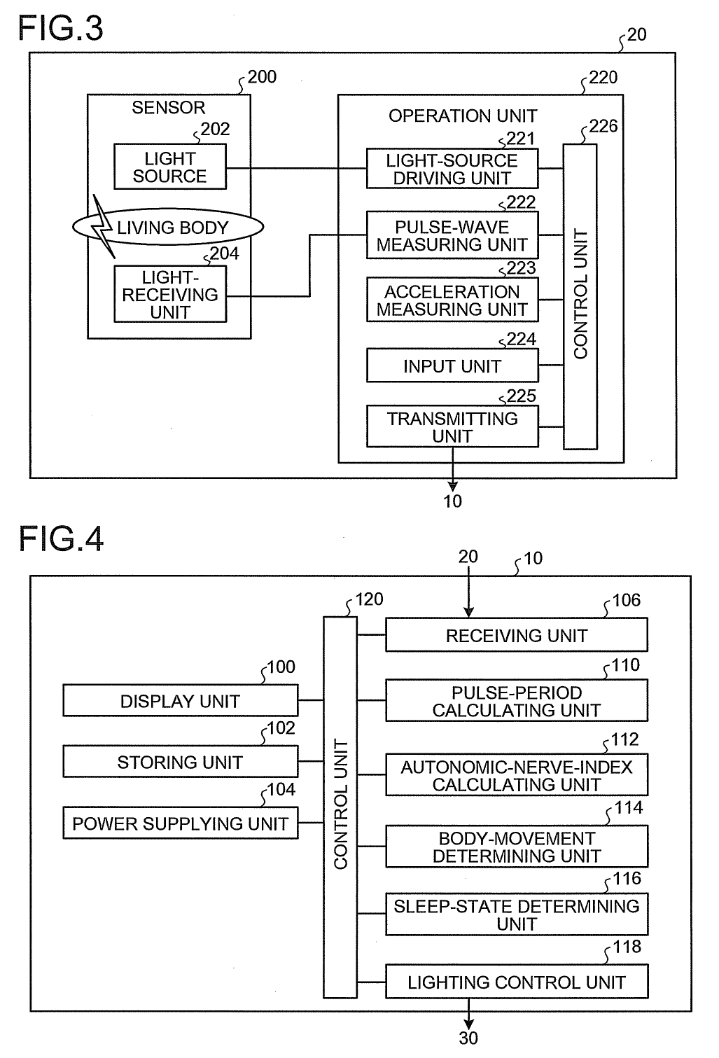

As shown in FIG. 3, the sensor 200 includes a light source 202 and a light-receiving unit 204. The light source 202 can be a blue LED, and a light-receiving unit 204 can be a photodiode. The light source 202 irradiates light onto the skin of the user, and the light-receiving unit 204 detects the light reflected from the skin of the user. The amount of light reflected from the skin varies as the amount of blood flowing in the capillaries under the skin varies.

The operation unit 220 includes a light-source driving unit 221, a pulse-wave measuring unit 222, an acceleration measuring unit 223, an input unit 224, a transmitting unit 225, and a control unit 226.

The light-source driving unit 221, which serves as a voltage supplying unit, drives the light source 202.

The pulse-wave measuring unit 222 measures the user's pulse wave and converts the pulse wave, which is analog data, into digital data. Specifically, the pulse-wave measuring unit 222 includes a current-voltage converter (not shown) that converts an output current from the light-receiving unit 204 into a voltage, and an amplifier (not shown) that amplifies the voltage. Furthermore, the pulse-wave measuring unit 222 includes a highpass filter (not shown) having a cutoff frequency of 0.1 hertz that highpass-filters the voltage, and a lowpass filter (not shown) having a cutoff frequency of 50 hertz) that lowpass-filters the voltage. Moreover, the pulse-wave measuring unit 222 includes a 10-bit A/D converter (not shown) that converts the resultant voltage, which is analog voltage, into digital data. The pulse-wave measuring unit 222 outputs the digital data to the control unit 226.

The acceleration measuring unit 223, which is an acceleration sensor, measures accelerations of the user's body and converts the accelerations, which are analog acceleration data, into digital data. Specifically, the acceleration measuring unit 223 measures accelerations in three-axis directions (hereinafter, “three-axis accelerations”) in a range of −2 g (gee or grav) to +2 g. The acceleration measuring unit 223 includes an adjustment circuit (not shown) that adjusts gain and offset of the analog acceleration data, and a 10-bit A/D converter (not shown) that converts the adjusted analog data into the digital data. The acceleration measuring unit 223 outputs the digital data to the control unit 226.

The input unit 224 is a device for inputting instructions. By operating the input unit 224, the user can turn on or off the sleep sensor 20. Moreover, by operating the input unit 224, the user can input an instruction to change the display contents.

The transmitting unit 225 transmits the digital data measured by the pulse-wave measuring unit 222 and that measured by the acceleration measuring unit 223 to the lighting control apparatus 10.

As shown in FIG. 4, the lighting control apparatus 10 includes a display unit 100, a storing unit 102, a power supplying unit 104, a receiving unit 106, a pulse-period calculating unit 110, an autonomic-nerve-index calculating unit 112, a body-movement determining unit 114, a sleep-state determining unit 116, a lighting control unit 118, and a control unit 120.

The display unit 100, which is, for example, liquid crystal display (LCD), displays a determination result of the user's sleep state. The storing unit 102 stores therein measured data received from the sleep sensor 20 such as the pulse wave data, electrocardiographic data, and body movement data, data obtained by processing the measured data such as the pulse period data, and such data as thresholds used for determining the user's sleep state. The storing unit 102 is, for example, a flash memory. The power supplying unit 104, which is, for example, a battery, is a power supply that supplies power to the lighting control apparatus 10. The receiving unit 106 receives data from the sleep sensor 20. The control unit 120 controls measurement timings of an electrocardiogram and the pulse wave, and controls accumulation and processing of the received data.

The pulse-period calculating unit 110 calculates a pulse period from the pulse wave measured by the pulse-wave measuring unit 222. The pulse-period is the time required to complete one cycle of the pulse wave.

Specifically, the pulse-period calculating unit 110 samples the pulse wave data obtained by the pulse-wave measuring unit 222, subjects the sampled pulse-wave data to time differential thereby obtaining direct-current (DC) fluctuation components of the pulse wave data. The pulse-period calculating unit 110 removes the obtained DC fluctuation component from the pieces of pulse wave data.

Moreover, the pulse-period calculating unit 110 acquires a maximum and a minimum of the pulse wave data for about one second before and after a processing time point at which the DC fluctuation components are removed from the pulse wave data. The pulse-period calculating unit 110 sets a value between the maximum and the minimum as a threshold. As the threshold, it is preferable to use, for example, a value having a 90% amplitude relative to the minimum, where the amplitude is the difference between the maximum and the minimum.

Furthermore, the pulse-period calculating unit 110 calculates time points at each of which the pulse wave data from which the DC fluctuation component is removed corresponds to the threshold appears. The pulse-period calculating unit 110 sets the time duration between the calculated time points as the pulse period.

The pulse period data is irregular data, i.e., pulse period data is not obtained at regular intervals. To perform frequency analysis on the pulse period data, it is necessary to convert the irregular data into regular data. The irregular pulse-period data is subjected to interpolation and re-sampling thereby generating regular pulse-period data. More specifically, the regular pulse-period data is generated using three sampling points, i.e., an interpolation-target point and two points before and after the interpolation-target point by cubic-polynomial interpolation.

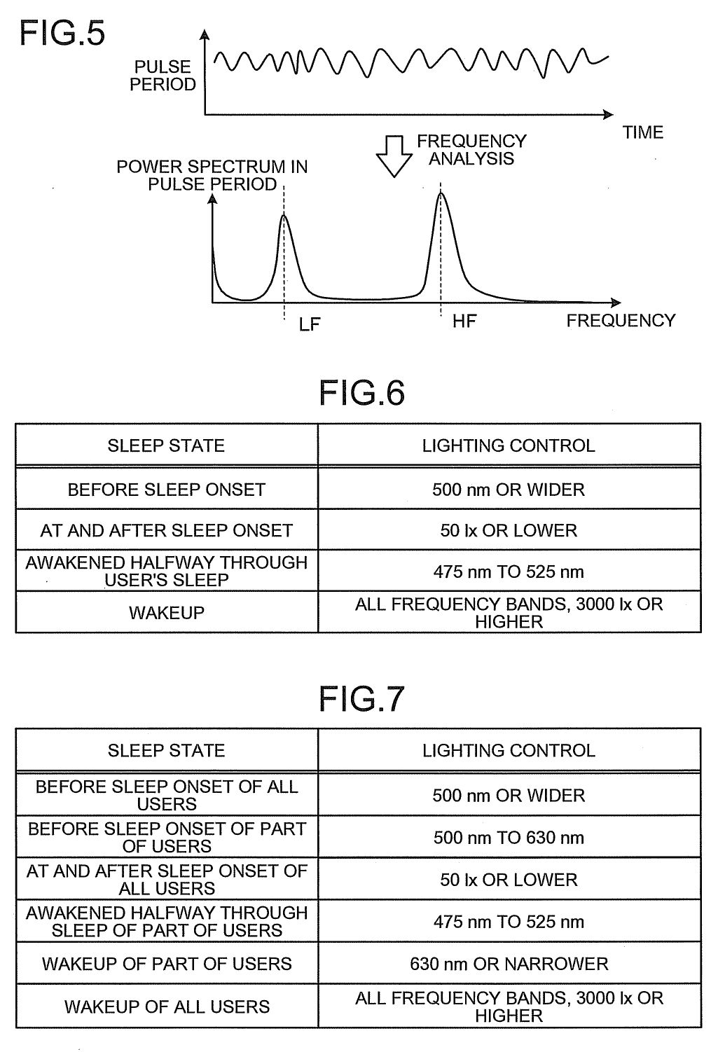

The autonomic-nerve-index calculating unit 112 calculates two autonomic nerve indexes, i.e., an index LF in a low frequency range, i.e., between about 0.05 hertz and about 0.15 hertz, and an index HF in a high frequency range, i.e., between about 0.15 hertz and about 0.4 hertz. FIG. 5 is an explanatory view of a processing performed by the autonomic-nerve-index calculating unit 112.

The autonomic-nerve-index calculating unit 112 transforms the regular pulse-period data into, for example, a frequency spectrum distribution by fast Fourier transform (FFT). The autonomic-nerve-index calculating unit 112 obtains the indexes LF and HF from the frequency spectrum distribution. More specifically, the autonomic-nerve-index calculating unit 112 calculates the indexes LF and HF each by averaging a peak of each of a plurality of power spectrums and two points equidistant to the peak.

The FFT is preferable because it lessens data processing burden. It is needless to say that other techniques, such as an auto regressive (AR) model method, a maximum entropy method (MEM), a wavelet method or the like, can be used instead of the FFT.

The body-movement determining unit 114 subjects the three-axis acceleration data received from the acceleration measuring unit 223 to time differential thereby obtaining differential coefficients of the respective three-axis accelerations. Then, the body-movement determining unit 114 calculates fluctuations in the body-movement data and body-movement amount. The fluctuations in the body-movement data are the root sum square of the differential coefficients of the respective three-axis accelerations. On the other hand, the body-movement amount is an average of the fluctuation in the body-movement data within the pulse period. If the body movement amount is larger than a first threshold, the body-movement determining unit 114 determines that the user's body is moving. The first threshold is, for example, a minimum of a fine body movement, i.e., 0.01 G, that can be measured with a body movement meter.

The sleep-state determining unit 116 determines that the user is in an awake state if the occurrence frequency of the body movement (hereinafter “body-movement occurrence frequency”) determined by the body-movement determining unit 114 is equal to or higher than a second threshold. The sleep-state determining unit 116 determines that the user is in an asleep state if the body-movement occurrence frequency is lower than the second threshold.

Specifically, the sleep-state determining unit 116 acquires a determination result, as to whether the user's body is moving, from the body-movement determining unit 114, and measures the body-movement occurrence frequency for a predetermined duration. For example, the predetermined duration is one minute. If the body-movement occurrence frequency is equal to or higher than the second threshold, the sleep-state determining unit 116 determines that the user is in the awake state. If the body-movement occurrence frequency is lower than the second threshold, the sleep-state determining unit 116 determines that the user is in the asleep state. For example, the second threshold is 20 times/minute based on the previous body-movement occurrence frequency in the user's awake state.

The sleep-state determining unit 116 further determines the sleep depth of the user for determining the sleep state based on the indexes LF and HF calculated by the autonomic-nerve-index calculating unit 112 and the determination result as to whether the user's body is moving. The sleep depth is an index indicating the degree of an active state of the user's brain. In the embodiment, the sleep-state determining unit 116 determines which state the user's asleep state corresponds to, the non-REM sleep state or the REM sleep state. Furthermore, the sleep-state determining unit 116 determines which sleep the non-REM sleep state of the user corresponds to, light sleep or deep sleep if the user's asleep state is determined as the non-REM sleep state.

The lighting control unit 118 controls the intensity and the wavelength of the light irradiated from the lighting device 30. Specifically, the lighting control unit 118 controls the light irradiated from the lighting device 30 according to the user's sleep state as follows.

The lighting control exercised when one user is sleeping in the bedroom is shown in FIG. 6. For example, before sleep onset of the user, the lighting control unit 118 controls the lighting device 30 to irradiate light in a waveband equal to or wider than 500 nanometers. At and after sleep onset, the lighting control unit 118 controls the lighting device 30 to irradiate light at an illuminance equal or lower than 50 luces. In the awakened state halfway through his/her sleep, the lighting control unit 118 controls the lighting device 30 to irradiate light in a waveband between 475 nanometers and 525 nanometers. At wakeup time, the lighting control unit 118 controls the lighting device 30 to irradiate light in all frequency bands at an illuminance equal to or higher than 3000 luces.

A lighting control exercised when a plurality of users are sleeping in the bedroom is shown in FIG. 7. Before sleep onset of all users present in the bedroom, the lighting control unit 118 controls the lighting device 30 to irradiate light in the waveband equal to or wider than 500 nanometers. Before sleep onset of a part of the users, the lighting control unit 118 controls the lighting device 30 to irradiate light in a waveband between 500 nanometers and 630 nanometers. At and after sleep onset of all the users, the lighting control unit 118 controls the lighting device 30 to irradiate light at an illuminance equal to or lower than 50 luces. In the awakened state halfway through his/her sleep of at least one user, the lighting control unit 118 controls the lighting device 30 to irradiate light in a waveband between 475 nanometers and 525 nanometers. At wakeup time of a part of the users, the lighting control unit 118 controls the lighting device 30 to irradiate light in a waveband equal to or narrower than 630 nanometers. At wakeup time of all the users, the lighting control unit 118 controls the lighting device 30 to irradiate light in all frequency bands at an illuminance equal to or higher than 3000 luces.

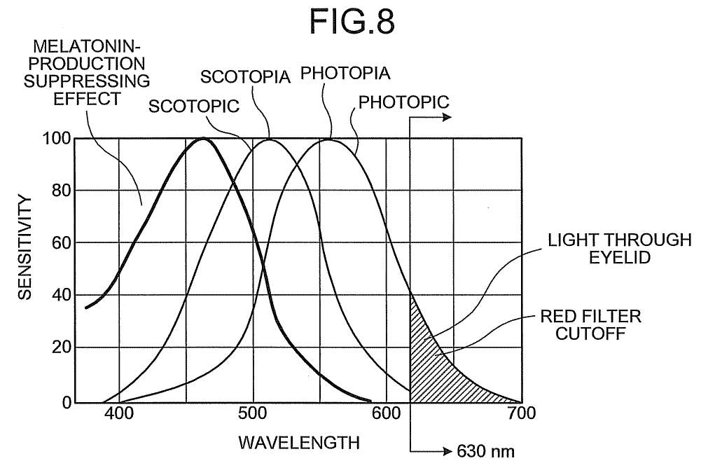

The relationship between the wavelength of light and human sensitivity to light is shown in the graph of FIG. 8. In FIG. 8, the horizontal axis indicates wavelength and the vertical axis indicates sensitivity.

The sensitivity relates to the photopia (light adaptation), the scotopia (dark adaptation), and the melatonin-production suppressing effect. The photopia is process by which a human eye adapts to an increase in illumination and by which the eye is sensible of light at a light location. The wavelength corresponding to the photopia is near 550 nanometers. The scotopia is process by which the eye adapts to a reduction in illumination and by which the eye is sensible of darkness at a dark location. The wavelength corresponding to the scotopia is near 500 nanometers.

Moreover, it is known that, if the production of melatonin is suppressed, the sleep onset is disturbed and the person suffers from insomnia. Accordingly, light at wavelengths at which the melatonin-production suppressing effect is great is not suited for the sleep onset. A peak of the wavelengths with the high melatonin-production suppressing effect is near 460 nanometers, and the melatonin-production suppressing effect is low at wavelengths equal to or wider than 500 nanometers.

Furthermore, the eye can sense light in the visible light range (630 nanometers to 780 nanometers) through the eyelid, i.e., even when the eye is in closed state. Therefore, if the light in the visible light range is irradiated on the user who is asleep, the user often feels dazzled and is awakened.

Using the above properties, therefore, before sleep onset of the user, the lighting device 30 is controlled to irradiate light in the waveband equal to or wider than 500 nanometers, which has smaller melatonin-production suppressing effect, as shown in FIG. 6. In the awakened state halfway through the sleep, the lighting device 30 is controlled to irradiate light in a waveband between 475 nanometers and 525 nanometers, which has smaller melatonin-production suppressing effect and which corresponds to the scotopia for the following reasons. If production of the melatonin is suppressed when the user is awakened halfway through his/her sleep, it undesirably takes time for the user to return to sleep. Furthermore, when the user is awakened halfway through his/her sleep, the user's eye is scotopic (dark-adapted). Therefore, the user's eye is sensible of light in the waveband corresponding to the scotopia as bright light. Moreover, the user in his/her sleep is insensible of the light in the waveband corresponding to the scotopia through the eyelid. Due to this, even if the other user who is in his/her sleep is present in the same bedroom, the light does not disturb the other user's sleep.

For these reasons, when the user is awakened halfway through his/her sleep, the lighting device 30 is controlled to irradiate the light with smaller melatonin-production suppressing effect and corresponding to the dark adaptation. Moreover, at and after sleep onset, the lighting device 30 is controlled to irradiate light at luminance equal to or lower than 50 luces. It is known that the user can easily go to sleep not in the environment of total darkness but in the environment of dim light. Alternatively, at and after sleep onset, the lighting device 30 can be controlled not to irradiate any light.

Furthermore, as shown in FIG. 7, before sleep onset of all the users, the lighting device 30 is controlled to irradiate light in the waveband equal to or wider than 500 nanometers, which has smaller melatonin-production suppressing effect. Before sleep onset of a part of the users, if the users are sensible of light through the eyelids, the light disturbs sleep of part of the users before sleep onset. For this reason, before sleep onset of a part of the users, the lighting device 30 is controlled not to irradiate light in the waveband wider than 630 nanometers of which light the users are sensible through the eyelids. Namely, before sleep onset of part of the users, the lighting device 30 is controlled to irradiate light in the waveband between 500 nanometers and 630 nanometers. It is to be noted that, even if the light in the waveband between 500 nanometers and 630 nanometers is irradiated, the light does not disturb users' sleep.

At and after sleep onset of all the users, the lighting device 30 is controlled to irradiate light at the illuminance equal to or lower than 50 luces similarly to FIG. 6. In the awakened state halfway through his/her sleep of at least one user, the lighting device 30 is controlled to irradiate light in the waveband between 475 nanometers and 525 nanometers corresponding to the dark adaptation. At wakeup time of a part of the users, the lighting device 30 is controlled to irradiate light in the waveband equal to or narrower than 630 nanometers, of which light the users are insensible through the eyelids because of presence of other users still in their sleep.

An incandescent lamp or a fluorescent lamp has been generally employed as the lighting device in the bedroom. The light discharged from the incandescent lamp has a wide wavelength distribution, and the fluorescent lamp uses phosphor that irradiates RGB light in response to ultraviolet rays. Due to these facts, the conventional lighting device expresses color by color mixture. As a result, the physiological action of the waveband of the light cannot be conventionally made active use of. The lighting device 30, by contrast, includes LEDs and can irradiate lights at different wavelengths. Therefore, the lighting device 30 can not only irradiate the light at an appropriate intensity for the sleep state of the user but also at an appropriate wavelength for the sleep state of the user.

In this manner, the lighting control system 1 enables the lighting control apparatus 10 to exercise irradiation control over the lighting device 30 so as not to degrade the sleep quality of each user by controlling the lighting device 30 to irradiate the light at the wavelength and the intensity appropriate for the sleep state of each user.

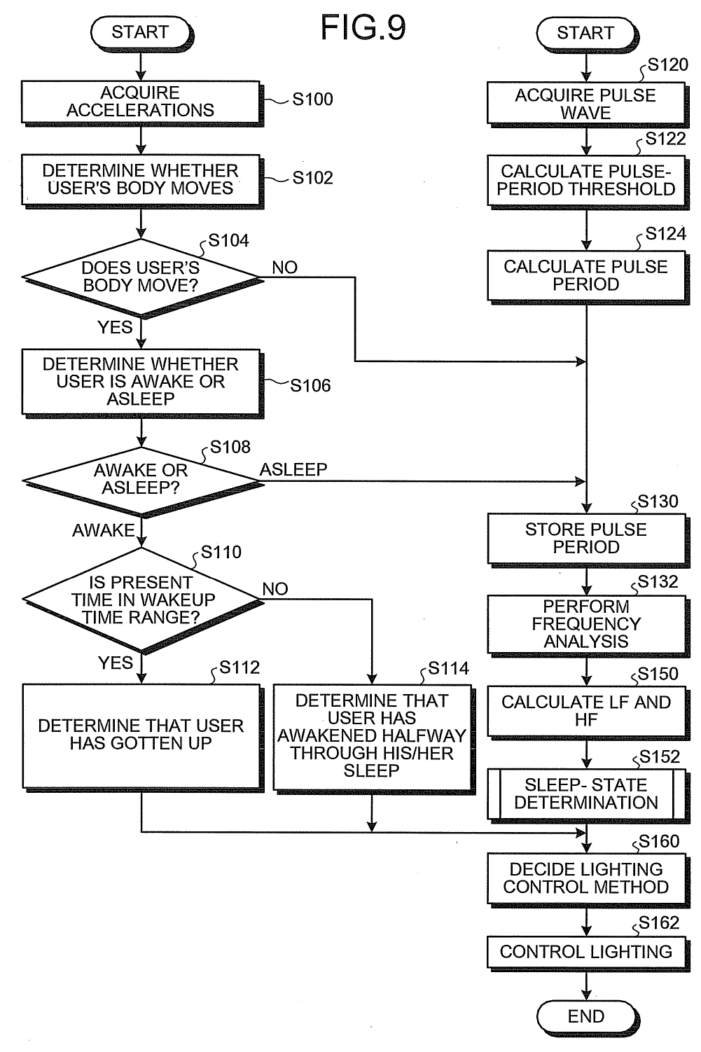

A lighting control processing performed by the lighting control system 1 will next be explained with reference to FIG. 9. Before going to bed, the user attaches the sleep sensor 20 to himself/herself, or “wears” the sleep sensor 20, and turns on the sleep sensor 20 by operating the input unit 224. The user also sets a wakeup time range by operating the input unit 224. The wakeup time range means a range, for example, from seven o'clock to seven thirty, including planned wakeup time. The wakeup time range can be set to a range of 15 minutes before and after the planned wakeup time or a range from 30 minutes before the planned wakeup time to the planned wakeup time. The wakeup time range can be set as desired. When the settings have been made, the acceleration measuring unit 223 of the sleep sensor 20 starts measuring accelerations and the pulse-wave measuring unit 222 starts measuring the pulse wave.

The acceleration measuring unit 223 of the sleep sensor 20 starts measuring accelerations and sends the measured accelerations to the receiving unit 106 of the lighting control apparatus 10 via the transmitting unit 225. The receiving unit 106 thereby acquires the measured accelerations (step S100). The body-movement determining unit 114 of the lighting control apparatus 10 acquires body movement data from the three-axis-direction acceleration data acquired from the acceleration measuring unit 223. If the fluctuation in the body-movement data is larger than the first threshold, the body-movement determining unit 114 determines that the user's body is moving (step S102).

If the body-movement determining unit 114 determines that the user's body is moving (Yes at step S104), the sleep-state determining unit 116 of the lighting control apparatus 10 determines whether the user is awake or asleep (step S106). If the sleep-state determining unit 116 determine that the user is awake (step S108; AWAKE), the sleep-state determining unit 116 further determines whether the present time is within the wakeup time range (step S110). If the present time is within the wakeup time range (Yes at step S110), the sleep-state determining unit 116 determines that the user has gotten up (step S112). If the present time is not within the wakeup time range (No at step S110), the sleep-state determining unit 116 determines that the user is awake halfway (step S114).

On the other hand, the pulse-wave measuring unit 222 of the sleep sensor 20 sends the measured pulse wave data to the receiving unit 106 of the lighting control apparatus 10 via the transmitting unit 225 (step S120). The pulse-period calculating unit 110 calculates the pulse-period threshold that is a dynamic threshold for calculating the pulse period (step S122). The pulse-period calculating unit 110 calculates time points at which the pulse wave data corresponding to the pulse-period threshold appears from the series of pulse wave data from which the DC fluctuation components are removed. In addition, the pulse-period calculating unit 110 obtains the time interval of the calculated time points as the pulse period (step S124).

Next, the pulse-period calculating unit 110 stores the pulse period data based on the result of the body-movement determination at the step S102 and the result of sleep/awake determination at the step S104 only when the user is asleep and the user's body does not move (step S130).

The pulse-period calculating unit 110 transforms the pulse period data into the frequency spectrum distribution by the frequency analysis such as the FFT (step S132). The autonomic-nerve-index calculating unit 112 calculates the indexes LF and HF from the power spectrums of the frequency spectrum distribution obtained at the step S132 (step S150). The sleep-state determining unit 116 performs a sleep-state determination processing thereby determining the sleep state of the user based on the autonomic nerve indexes LF and HF, and stores the determination result in the storing unit 102 (step S152).

The lighting control unit 118 of the lighting control apparatus 10 decides a method of controlling the lighting device 30 according to the sleep state of the user (step S160). The method of controlling the lighting device 30 is decided according to the control processing explained with reference to FIG. 6 or 7. The lighting control unit 118 exercises the decided lighting control over the lighting device 30 (step S162). The lighting control processing is thereby finished.

If the lighting control apparatus 10 is to be used by a plurality of users, the sleep sensor 20 is attached to each of the users. The lighting control apparatus 10 acquires the acceleration data and the pulse wave data from the sleep sensor 20 of each user, and determines the sleep state of each user.

Moreover, if the lighting control unit 118 acquires the acceleration data and the pulse wave data only from one sleep sensor 20, then the lighting control unit 118 determines that only one user is going to sleep in the bedroom and performs the control processing shown in FIG. 5. If the lighting control unit 118 acquires the acceleration data and the pulse wave data from a plurality of sleep sensors 20, then the lighting control unit 118 determines that a plurality of users is going to sleep in the bedroom and performs the control processing shown in FIG. 6.

Alternatively, information on the number of users who are going to sleep in the bedroom can be input to the sleep sensor 20 attached to each user from the input unit 224 of the sleep sensor 20. In this case, the information on the number of users who are going to sleep in the bedroom is transmitted from the transmitting unit 225 of the sleep sensor 20 to the receiving unit 106 of the lighting control apparatus 10. The lighting control unit 118 determines whether one user or a plurality of users is present in the bedroom based on the acquired information on the number of users, and performs the control processing based on the determination.

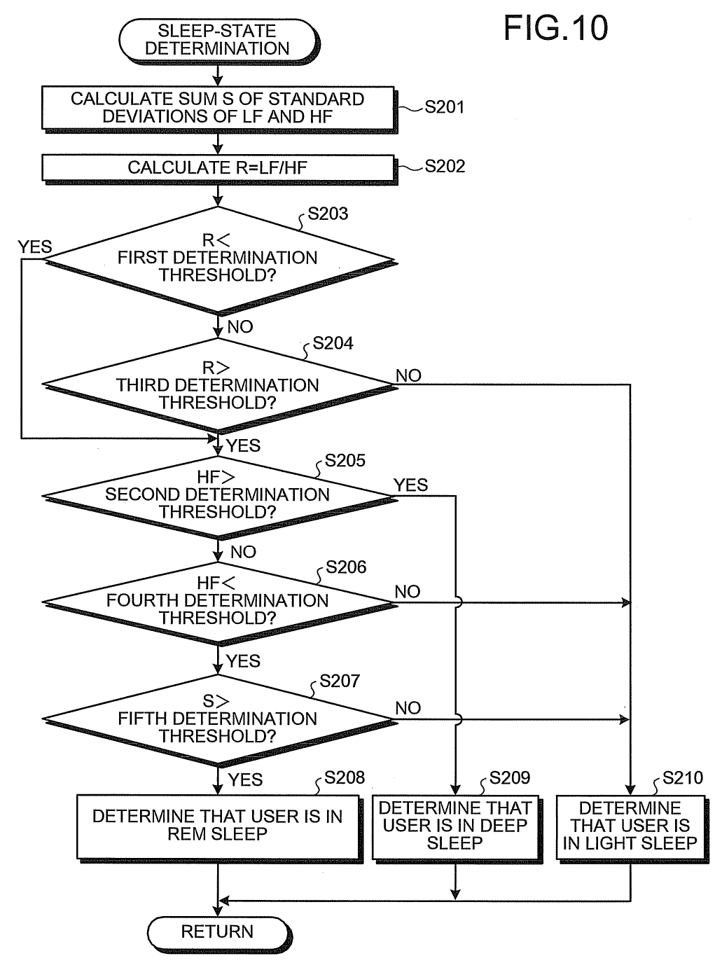

The sleep-state determination processing performed at the step S152 will be explained in detail with reference to FIG. 10. The sleep-state determining unit 116 acquires the indexes LF and HF from the autonomic-nerve-index calculating unit 112 and calculates the sum S of standard deviations of LF and HF (step S201). Furthermore, the sleep-state determining unit 116 calculates R, which is a ratio LF/HF (step S202).

The sleep-state determining unit 116 then determines whether R is lower than a first determination threshold (step S203). If R is lower than the first determination threshold (Yes at step S203), the sleep-state determining unit 116 further determines whether HF is greater than a second determination threshold (step S205). If HF is greater than the second determination threshold (Yes at step S205), the sleep-state determining unit 116 determines that the user is in deep sleep (step S209).

If R is equal to or lower than the first determination threshold (No at step S203), the sleep-state determining unit 116 further determines whether R is higher than a third determination threshold (step S204). If R is higher than the third determination threshold (Yes at step S204), the sleep-state determining unit 116 further determines whether HF is greater than the second determination threshold (step S205).

If HF is equal to or smaller than the second determination threshold (No at step S205), the sleep-state determining unit 116 further determines whether HF is smaller than a fourth determination threshold (step S206). If HF is smaller than the fourth determination threshold (Yes at step S206), the sleep-state determining unit 116 further determines whether S is greater than a fifth determination threshold (step S207). If S is greater than the fifth determination threshold (Yes at step S207), the sleep-state determining unit 116 determines that the user is in REM sleep (step S208).

If R is equal to or lower than the third determination threshold (No at step S204), if HF is equal to or greater than the fourth determination threshold (No at step S206), or if S is equal to or smaller than the fifth determination threshold (No at step S207), the sleep-state determining unit 116 determines that the user is in a light sleep (step S210).

The first to fifth determination thresholds can be set, for example, as follows. Two high-density points are selected from each of distributions of LF, HF, and R measured per user overnight. A midpoint of the two points selected from the distribution of R is set as both the first determination threshold and the third determination threshold. A midpoint of the two points selected from the distribution of HF is set as both the second determination threshold and the fourth determination threshold. Furthermore, a midpoint of the two points selected from the distribution of LF is set as the fifth determination threshold.

Because the three-axis-direction acceleration data is measured as the body movement data, it is possible to easily, accurately measure the body movement of the user. It is, therefore, possible to lessen the influence of the body movement on the pulse wave and that of abnormality in pulse wave such as arrhythmia or apnea on the pulse wave. Accordingly, it is possible to improve accuracy of determining the sleep state of the user.

It has been explained above that the lighting control apparatus 10 makes the asleep-state determination up to whether the asleep state of the user is the REM sleep state or the non-REM sleep state. However, if the sleep state of the user is determined as the asleep state, it is not always necessary to further determine whether the asleep state is the REM sleep state or the non-REM sleep state. It suffices to be able to determine which state the sleep state of the user correspond to, the state before sleep onset, the state at and after the sleep onset, the awakened state halfway through his/her sleep, and the wakeup state.

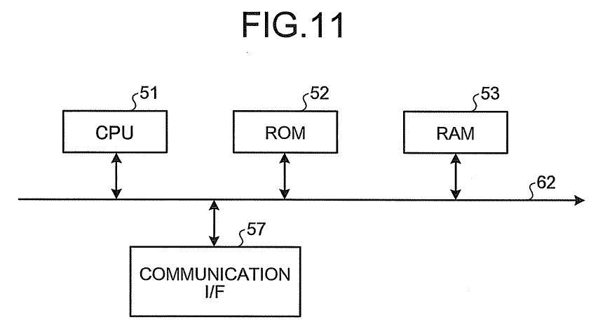

A hardware configuration of the lighting control apparatus 10 is shown in FIG. 11. The hardware configuration includes a read only memory (ROM) 52, a central processing unit (CPU) 51, a random access memory (RAM) 53, a communication interface (I/F) 57, and a bus 62. The ROM 52 stores therein a computer program (hereinafter, “lighting control program”) for causing the lighting control apparatus 10 to perform the lighting control processing. The CPU 51 controls the ROM 52, the RAM 53, and the communication I/F 57 according to the lighting control program. The RAM 53 stores therein various data necessary for the lighting control apparatus 10 to exercise lighting control over the lighting device 30. The communication I/F 57 connects the lighting control apparatus 10 to a network (not shown) to establish communication. The bus 62 connects the ROM 52, the CPU 51, the RAM 53, and the communication I/F 57 to one another.

Alternatively, the lighting control program can be recorded in a computer-readable recording medium (not shown) such as a CD-ROM, a floppy disk (FD) or a digital versatile disk (DVD) as a file in an installable form or executable form, and provided to the lighting control apparatus 10.

When the lighting control program is provided on the computer-readable recording medium, the lighting control program is loaded onto a main memory device (not shown) of the lighting control apparatus 10 by reading the lighting control program from the recording medium, and the software constituent elements shown in FIG. 4 are generated on the main memory device.

As another alternative, the lighting control program can be stored in another computer (not shown) connected to the network, such as the Internet, and the lighting control program can be downloaded into the lighting control apparatus 10.

Although the invention has been described with respect to the embodiment, various changes and modifications can be made of the invention.

In the embodiment, the autonomic nerve indexes HF and LF are calculated to determine the sleep state of the user. Alternatively, brain waves of the user can be measured instead of calculating the indexes HF and LF. If the brain waves are measured, the sleep state of the user is determined based on an analysis result of the alpha wave, the beta wave, and the gamma wave of the user. As another alternative, a user's electrocardiogram can be measured to determine the sleep state of the user. In this case, the sleep state of the user is determined based on a status of autonomic nervous activities read from the electrocardiogram. As still another alternative, a user's heartbeat can be measured to determine the sleep state of the user. In this case, the sleep state of the user is determined based on the standard deviation from an average heartbeat.

Moreover, as yet another alternative, the heartbeat and respiration of the user can be measured to determine the sleep state of the user. It suffices to determine the sleep state of the user by analyzing at least one signal obtained from the calculation of the autonomic nerve indexes, the measurement of the brain waves, the measurement of the electrocardiogram, the measurement of the heartbeat, the measurement of the respiration and the like. In this manner, the measurement target for specifying the sleep state is not limited to that explained in the embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A lighting control apparatus comprising:
a detecting unit that detects a sleep state of a user; and
a control unit that controls a lighting device to irradiate a light in a waveband with a relatively small melatonin-production suppressing effect onto the user if the detecting unit detects a state before a sleep onset.

2. The apparatus according to claim 1, wherein the control unit controls the lighting device to irradiate a light in a waveband equal to or wider than 500 nanometers more strongly than lights in wavebands other than the waveband equal to or wider than 500 nanometers.

3. The apparatus according to claim 1, wherein the control unit controls the lighting device to irradiate a light in a waveband corresponding to scotopia if the detecting unit detects that the user is awakened halfway through user's sleep.

4. The apparatus according to claim 3, wherein the control unit controls the lighting device to irradiate a light in a waveband between 475 nanometers and 525 nanometers more strongly than lights in wavebands other than the waveband between 475 nanometers and 525 nanometers.

5. The apparatus according to claim 1, wherein the control unit controls the lighting device to irradiate a light at an illuminance equal to or lower than 50 luxes if the detecting unit detects a state at or after a sleep onset.

6. The apparatus according to claim 1, wherein the detecting unit detects sleep states of a plurality of users,
the control unit controls the lighting device to irradiate a light, of which the users are less sensible through eyelids of the users, in a waveband without the melatonin-production suppressing effect onto the users if at least one of the users is in states before the sleep onset and remaining users are asleep.

7. The apparatus according to claim 6, wherein the control unit controls the lighting device to irradiate a light in a waveband between 500 nanometers and 630 nanometers more strongly than lights in wavebands other than the waveband between 500 nanometers and 630 nanometers.

8. The apparatus according to claim 1, wherein the detecting unit detects sleep states of a plurality of users, and
the control unit controls the lighting device to irradiate a light of which the users are less sensible through eyelids of the users onto the user if the sleep-state detecting unit detects that at least one of the users is awake and that remaining users are asleep.

9. The apparatus according to claim 8, wherein the control unit controls the lighting device to irradiate a light in a waveband equal to or narrower than 630 nanometers more strongly than lights in wavebands other than the waveband equal to or narrower than 630 nanometers.

10. A lighting system comprising:
the lighting control apparatus according to claim 1; and
a lighting device that irradiates the light in a waveband with a relatively small melatonin-production suppressing effect onto the user.

11. The system according to claim 10, wherein the lighting device includes a plurality of light-emitting diodes.

12. The system according to claim 10, wherein the lighting device includes a laser diode.

13. The system according to claim 10, wherein the lighting device includes an optical filter.

14. A lighting control apparatus comprising:
a detecting unit that detects a sleep state of a user; and
a control unit that controls a lighting device to irradiate a light in a waveband corresponding to scotopia if the detecting unit detects that the user is awakened halfway through user's sleep.

15. A lighting control apparatus comprising:
a detecting unit that detects sleep states of a plurality of users; and
a control unit that controls a lighting device to irradiate a light of which the users are less sensible through eyelids of the users if the sleep-state detecting unit detects that at least one of the users is awake and that remaining users are asleep.

16. A method for lighting control comprising:
detecting a sleep state of a user; and
controlling a lighting device to irradiate a light in a waveband with a relatively small melatonin-production suppressing effect if a state before a sleep onset is detected at the detecting.

17. A computer program product comprising a computer-readable recording medium, the computer-readable recording medium executable by a computer and including a plurality of instructions for a lighting control processing, the instructions including
an instruction to detect a sleep state of a user; and
an instruction to control a lighting device to irradiate a light in a waveband with a relatively small melatonin-production suppressing effect if a state before a sleep onset is detected.
